(19) United States
(12) Patent Application Publication
(10) Pub. No.: US 2024/0274279 A1
(43) Pub. Date: Aug. 15, 2024

(54) Animal Treatment Clinic Information System and Method of Use

(71) Applicant: Nikki Gomas, Weatherford, TX (US)

(72) Inventor: Nikki Gomas, Weatherford, TX (US)

(21) Appl. No.: 18/479,159

(22) Filed: Oct. 2, 2023

Related U.S. Application Data
(60) Provisional application No. 63/445,467, filed on Feb. 14, 2023.

(51) Int. Cl. G16H 40/20

(52) U.S. Cl. CPC G16H 40/20

(57) ABSTRACT

A system and method for providing detailed information on nearby animal hospitals, veterinary clinics, and other animal treatment centers. The system provides a user-friendly website and mobile application enabling users to enter their zip code, one or more selection criteria for displaying veterinary hospitals and clinics within a certain radius of the user's location. The system provides users with comprehensive information about each veterinary hospital or clinic, including staff size, age of the business, types of services offered, estimated cost per visit, contact details, working hours, doctor profiles, reviews, and average rankings/ratings provided by registered users. This detailed information assists users in making informed decisions about selecting the most suitable hospital or clinic for their pets' needs. The system is a centralized platform with reliable and up-to-date information, thus eliminating the challenges pet owners face in finding and evaluating veterinary services.

### CROSS-REFERENCE TO RELATED APPLICATION

The present application claims priority to, and the benefit of, U.S. Provisional Application No. 63/445,467, which was filed on Feb. 14, 2023 and is incorporated herein by reference in its entirety.

### FIELD OF THE INVENTION

The present invention generally relates to the field of selection of veterinary hospitals and clinics. More specifically, the present invention relates to a novel system and method capable of detailing information on nearby animal hospitals, veterinary clinics, and other animal treatment centers. The system, via a mobile application or a website, enables pet owners to obtain highly detailed information about each facility, such as staff size, age of business, services provided, average cost, reviews, contact information, and website links for making an informed decision in selection of veterinary services. Accordingly, the present disclosure makes specific reference thereto. Nonetheless, it is to be appreciated that aspects of the present invention are also equally applicable to other like applications, devices, and methods of manufacture.

### BACKGROUND

By way of background, pet owners often find themselves unaware of all the veterinary services and hospitals available near their home, and especially when they move to a new area. It is common for people in such situations to rely on asking friends and neighbors for recommendations or search online through various reviews and recommendations. However, this process can be challenging and time-consuming, as there is no dedicated and comprehensive source for veterinary information. Many times, pet owners end up going hospitals which are far from their home because of lack of information about nearby veterinary services and hospitals.

One of the common challenges pet owners face is the lack of a centralized and reliable platform specifically designed for veterinaries. Currently, it becomes difficult for pet owners to gather accurate and up-to-date information about nearby veterinary services and hospitals. Many of the details such hospital information and reviews are not updated and trustworthy which leads to selection of wrong veterinary.

Search engines and websites only provide general information about veterinary services. People are unable to find the specifics such as details about specific treatments offered by hospitals or clinics. Lack of specific information can make the selection process tedious and frustrating for pet owners who are seeking the best possible care for their beloved pets. Pet owners end up making wrong and uninformed decisions due to lack of a dedicated platform for veterinary medicine that can provide detailed and correct information.

Therefore, there exists a long felt need in the art for a system capable of identifying, storing, and providing detailed information on animal hospitals, veterinary clinics, and other animal treatment centers. Additionally, there is a long felt need in the art for a system that can provide information of nearby animal hospitals, veterinary clinics, and other animal treatment centers on their smartphones and other similar devices. Moreover, there is a long felt need in the art for a system that provides updated and genuine information about animal hospitals, veterinary clinics, and other animal treatment centers. Further, there is a long felt need in the art of a platform that provides highly detailed information about each veterinary facility. Furthermore, there is a long felt need in the art for an improved system that is dedicated for providing information about veterinary medicine to users. Finally, there is a long felt need in the art for a system that enables pet owners to make quick and informed decisions about selection of animal hospitals, veterinary clinics, and other animal treatment centers.

The subject matter disclosed and claimed herein, in one embodiment thereof, comprises veterinary hospitals and clinics detailed information providing system. The system further features a mobile application installed in a user device, a server system connected to the user device via Internet, the server system includes a web server configured to receive and process requests received from the mobile application, and to provide static and dynamic content to the mobile application about nearby veterinary hospitals, clinics, animal trainers, animal groomers, zoos, animal rehabs, and more, a recommendation module utilizes algorithms to provide personalized recommendations to users based on user preferences, location, and past interactions, a hospitals and clinic database for storing detailed information about animal hospitals and veterinary clinics, including clinic names, addresses, contact information, staff size, working hours, services offered, veterinarian profiles, customer reviews and rankings/ratings and a pets database securely storing pet information, including pet profiles and historical data, to aid in the recommendation of animal hospitals, veterinary clinics, and other animal treatment places. The system can be accessed using a web browser as well.

In this manner, the veterinary hospitals and clinics detailed information providing system and associated method of the present invention accomplish all of the forgoing objectives and provide users with a website or mobile application capable of detailing information on nearby animal hospitals, veterinary clinics, and other animal treatment centers. The system provides updated and genuine information such as staff size, age of business, services provided, average cost, reviews, contact information, telehealth contact information, and website links enabling pet owners to make informed decisions.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a veterinary hospitals and clinics information system. The system further comprising a mobile application installed in a user device, a server system connected to the user device through a network, the server system includes a web server configured to receive and process user requests, and to provide static and dynamic content to the mobile application, a recommendation module utilizes algorithms to provide personalized recommendations to users based on user preferences, location, and past interactions, a hospital and clinic database storing detailed information about animal hospitals and veterinary clinics, including clinic names, addresses, contact information, staff size, working hours, services offered, veterinarian profiles, telehealth contact information, customer reviews and rankings/ratings and a pets database securely storing pet information, including pet profiles and historical data, to aid in the recommendation of animal hospitals, veterinary clinics, and other animal treatment places.

In yet another embodiment, the mobile application is configured to display nearby veterinary hospitals and clinics, wherein a search bar is provided for inputting a veterinary hospital/clinic name or a service name for a search, a zip code search box for entering a zip code to search nearby veterinary hospitals and clinics, and the mobile application utilizes a location detection facility, such as a GPS module, to automatically detect the location of the user device and display a list of nearby veterinary hospitals and clinics on the user interface.

In yet another embodiment, the mobile application further displays a user interface displaying a hospital name, a staff size indicating the size of the hospital's staff, a life or age of the business of the hospital representing the duration of operations, types of services offered by the hospital, an estimated cost per visit or consultancy provided by the hospital, contact information including the hospital's website, telehealth contact, and email address, and an average ranking of the hospital.

In yet another embodiment, the mobile application is configured to display an interactive and digital map displaying a plurality of icons representing veterinary hospitals, clinics, and other centers.

In a further embodiment, a method for providing information on nearby veterinary hospitals and clinics is described. The method comprising the steps of receiving and validating a user-entered zip code in a mobile application and retrieving location information, including latitude and longitude, associated with the zip code; searching and displaying nearby veterinary hospitals and clinics within a certain radius using the obtained coordinates, and considering user profiles or preferences to narrow down the search results, receiving selection of one or more hospitals or clinics from the displayed search results by the user; and retrieving more detailed information about the selected hospital or clinic, including contact details, working hours, services offered, doctor profiles, reviews, and any other relevant data.

In a further embodiment, the method further comprising displaying a notification on the mobile application indicating a higher ranked and available veterinary hospital in the geographic range of a user upon selection of another veterinary hospital or clinic by the user, wherein the notification can be based upon ranking stored in the system or on a recommendation of one or more customers of another veterinary hospital.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of one potential embodiment of the veterinary hospitals and clinics information system of the present invention in accordance with the disclosed architecture;

FIG. 2 illustrates an exemplary user interface displayed by the mobile application and/or website for showing nearby veterinary hospitals and clinics to a user in accordance with the disclosed architecture;

FIG. 3 illustrates an exemplary digital map offered by the veterinary hospitals and clinics information system on the application and the website in accordance with the disclosed architecture;

FIG. 4 illustrates a user interface offered by the mobile application and website for displaying detailed information about a hospital/clinic in accordance with the disclosed architecture;

FIG. 5A illustrates another user interface allowing a user to choose one or more services for displaying veterinary professionals using the mobile application and/or website in accordance with the disclosed architecture;

FIG. 5B illustrates a perspective view displaying detailed information about a selected veterinary professional in accordance with the disclosed architecture;

FIG. 6 illustrates another user interface designed to provide different types of audio-video content to users such as owners of pets in accordance with the disclosed architecture;

FIG. 7 illustrates another user interface configured for displaying other types of services offered by the veterinary information system of the present invention in accordance with the disclosed architecture;

FIG. 8 illustrates a user interface offered by the animal hospitals and veterinary clinics information system allowing users to access and share digital documents of their pets with veterinary practitioners for consultation in accordance with the disclosed architecture;

FIG. 9 illustrates a flow diagram depicting a process of displaying detailed information of relevant veterinary hospitals and clinics to a user in accordance with one embodiment of the present invention;

FIG. 10 illustrates another flow diagram depicting a process of displaying detailed information of relevant veterinary hospitals and clinics to a user in accordance with one embodiment of the present invention; and

FIG. 11 illustrates a flow diagram depicting a process of receiving care sheets and recommendations from a veterinary practitioner in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a system capable of identifying, storing, and providing detailed information on animal hospitals, veterinary clinics, and other animal treatment centers. Additionally, there is a long felt need in the art for a system that can provide nearby animal hospitals, veterinary clinics, and other animal treatment centers on their smartphones and other similar devices. Moreover, there is a long felt need in the art for a system that provides updated and genuine information about animal hospitals, veterinary clinics, and other animal treatment centers. Further, there is a long felt need in the art of a platform that provides highly detailed information about each veterinary facility. Furthermore, there is a long felt need in the art for an improved system that is dedicated for providing information about veterinarians to users. Finally, there is a long felt need in the art for a system that enables pet owners to make quick and informed decisions about selection of animal hospitals, veterinary clinics, animal trainers, animal groomers, and other animal treatment centers.

The present invention, in one exemplary embodiment, is a method for providing information on nearby veterinary hospitals and clinics. The method comprising the steps of receiving and validating a user-entered zip code in a mobile application and retrieving location information, including latitude and longitude, associated with the zip code; searching and displaying nearby veterinary hospitals and clinics within a certain radius using the obtained coordinates, and considering user profiles or preferences to narrow down the search results, receiving selection of one or more hospitals or clinics from the displayed search results by the user; and retrieving more detailed information about the selected hospital or clinic, including contact details, telehealth details, working hours, services offered, doctor profiles, reviews, and any other relevant data.

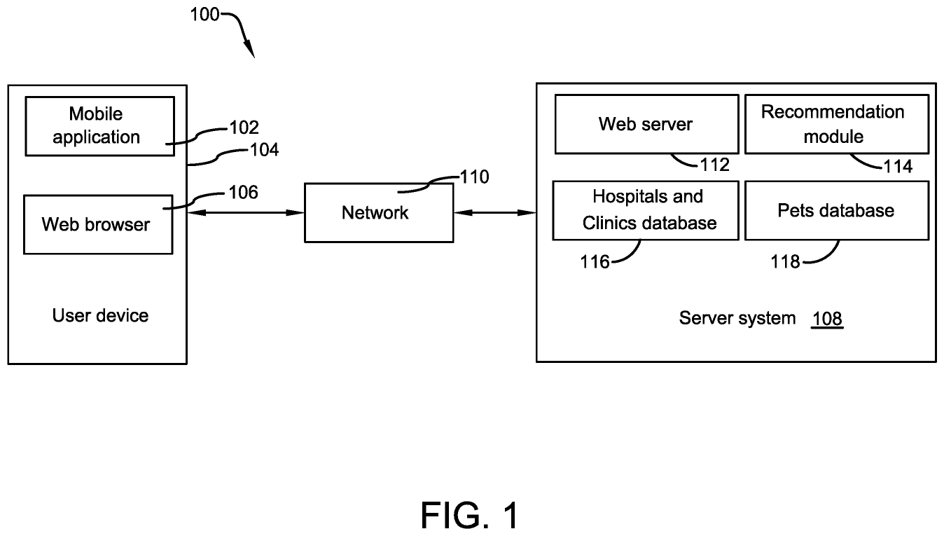

Referring initially to the drawings, FIG. 1 illustrates a schematic view of one potential embodiment of the veterinary hospitals and clinics information system of the present invention in accordance with the disclosed architecture. The veterinary hospitals and clinics information system 100 of the present invention is configured to provide users with a website or software application capable of detailing information on nearby animal hospitals, veterinary clinics, and other animal treatment centers. The system 100 includes a mobile application 102 installed in a user device 104. A user interacts with the system 100 using the user device 104, which can be a smartphone, tablet, or any other internet-enabled electronic device. The user can use the installed mobile application 102 or the web browser 106 to access the services provided by a server system 108. This enables users to access the system 100 on various platforms.

The user device 104 and a server system 108 are connected through a network 110. The network 110 is preferably a public network such as the internet but can also be a private network or a cellular communications network or the like. Communication to the network 110 can be achieved through the use of a network service provider. The network 110 enables data transfer between the user device 104 and the server system 108, ensuring seamless communication between the user and the system 100. The server system 108 is configured for handling user requests and providing the requested information. A web server 112 is configured to receive and process user requests. The web server 112 also provides the required information to the users. More specifically, the web server 112 can provide static data including HTML, CSS, JavaScript, images and more and dynamic content to the mobile application 102 and the web browser 106. In the preferred embodiment, the web server 112 can support server-side technologies like PHP, Python, Node.js, or Java, enabling the execution of scripts and processing of dynamic web pages. The server system 108 can be a network of computers, a software framework, or a combination thereof, that may provide a generalized approach to create the server implementation. The communication network 110 can include suitable logic, circuitry, and interfaces that may be configured to provide a plurality of network ports and a plurality of communication channels for transmission and reception of data related to operations of various entities (such as the user device 104 and the server system 108) of the system 100.

A recommendation module 114 is configured to utilize algorithms for providing personalized recommendations to users. The recommendation module 114 can use user preferences, location, past interactions, and more for suggesting animal hospitals, veterinary clinics, animal trainers, and animal groomers meeting the user's requirements. A hospitals and clinic database 116 is designed to store detailed information about animal hospitals and veterinary clinics. The hospitals and clinic database 116 can include data including but not limited to clinic names, addresses, contact information, telehealth details, staff size, working hours, services offered, veterinarian profiles, customer reviews and rankings/ratings, and other relevant information necessary for users to make informed decisions for selection of a clinic for their pets. A pets database 118 is designed to securely store pet information. The database 118 can include profiles of pets, historical data, and potentially other relevant details that helps the recommendation module 114 to recommend specific animal hospitals, veterinary clinics, animal trainers, animal groomers, and other animal treatment places.

The communication between the user device 104 and the server system 108 can be performed via the communication protocols including but not limited to, Hypertext Transfer Protocol (HTTP), File Transfer Protocol (FTP), Simple Mail Transfer Protocol (SMTP), Domain Network System (DNS) protocol, Common Management Interface Protocol (CMIP), Transmission Control Protocol and Internet Protocol (TCP/IP), User Datagram Protocol (UDP), Long Term Evolution (LTE) communication protocols, or any combination thereof.

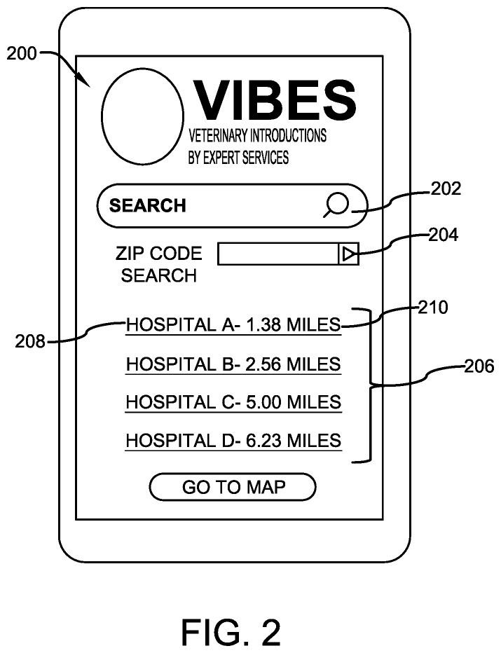

FIG. 2 illustrates an exemplary user interface displayed by the mobile application and/or website for showing nearby veterinary hospitals and clinics to a user in accordance with the disclosed architecture. The mobile application 102 and the website 106 are configured to display different user interfaces allowing users to interact with the system. In the present embodiment, the mobile application 102 displays nearby veterinary hospitals and clinics. As illustrated, the user interface 200 provides a search bar 202 enabling a user to input a veterinary hospital/clinic name or a service name for search. For allowing a user to search nearby a zip code, the zip code search box 204 is provided. Any zip code can be entered in the search box 204 and the user interface 200 can display a list 206 of veterinary hospitals and clinics.

In some embodiments of the present invention, the mobile application 102 is configured to use location detection facility such as GPS module of the user device 104 in which the application 102 is installed to automatically detect location of the device 104. Based on the detected location, a list of nearby veterinary hospitals and clinics is displayed on the user interface 200. Along with the hospitals and clinics names 208, a relative distance 210 from the detected or input location is also displayed for enabling a user to make an informed decision. The distance can be in any unit including miles, kilometers, meters, and more.

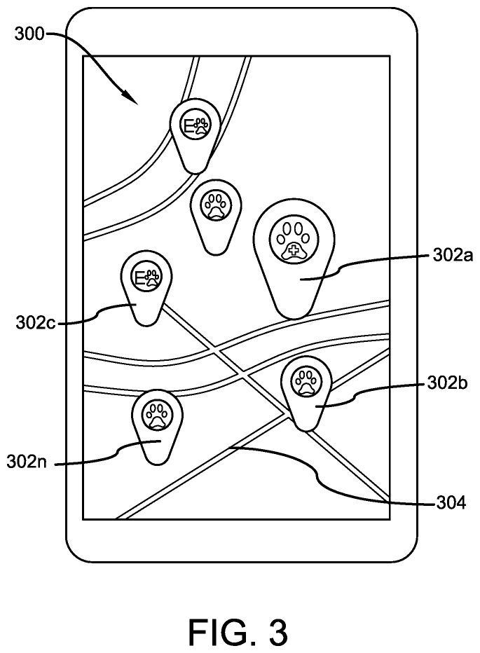

The system 100 enables the application 102 and the website 106 to display the veterinary hospitals and clinics on an interactive and digital map as illustrated in FIG. 3. For displaying one or more veterinary hospitals, clinics or other centers on a map, the “Go To Map” icon 212 can be selected to display the map as illustrated in FIG. 3.

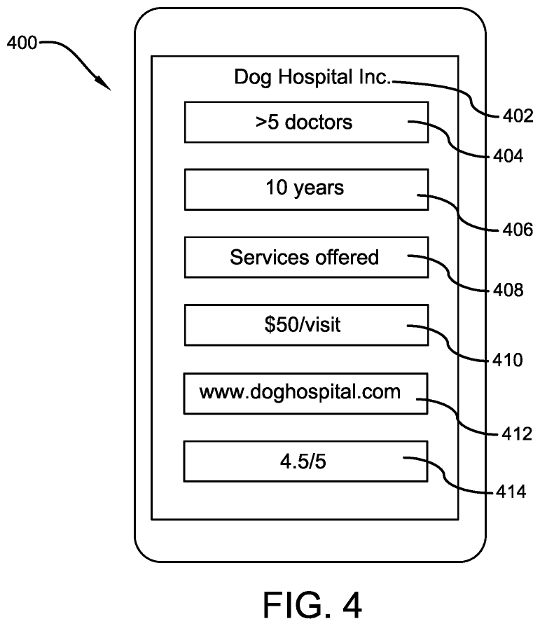

FIG. 3 illustrates an exemplary digital map offered by the veterinary hospitals and clinics information system on the application and the website in accordance with the disclosed architecture. As illustrated, the digital map 300 displays different veterinary hospitals, clinics, and trainers/groomers in the form of icons 302a-n such that each icon is selectable enabling a user to view detailed information thereof as illustrated in FIG. 4. The map 300 also displays real time distance between real time location or a predetermined location 304 of a user using the application 102 and/or website 104.

In some embodiments, an average rating of individual veterinary hospitals, clinics, and trainers/groomers is also displayed along with the corresponding icon helping a user to make an informed decision in selecting a hospital, clinic, or any other pet center.

FIG. 4 illustrates a user interface offered by the mobile application and website for displaying detailed information about a hospital/clinic in accordance with the disclosed architecture. The user interface 400 can be displayed upon selection of an icon in FIG. 3 or can be directly displayed on searching a hospital name in the search bar 202 of FIG. 2. As illustrated, the user interface 400 displays a hospital name 402 for which the detailed information is displayed by the interface 400. The interface 400 displays staff size 404 of the hospital, life or age of the business of hospital 406, the types of services offered 408, estimated cost per visit or consultancy 410, contact information including website and email address 412 and an average ranking 414 given by the users registered on the system 100.

The detailed information provided on the interface 400 enables users to make an informed decision to visit the hospital/clinic for their pets. The detailed information eliminates the need to take guesses as to what type of services each clinic offers for animal treatments and thus makes the process faster, efficient, and accurate. Furthermore, the information is updated in real time and includes all the ranking of the hospitals, clinics, trainers, groomers, and more from the system 100 and also from third party sources such as regulated databases.

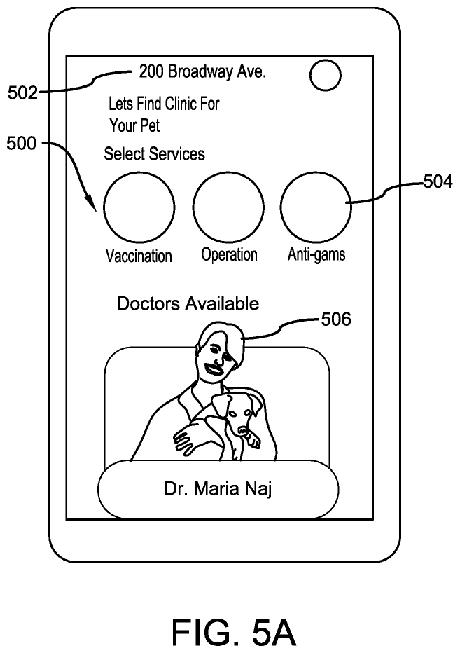

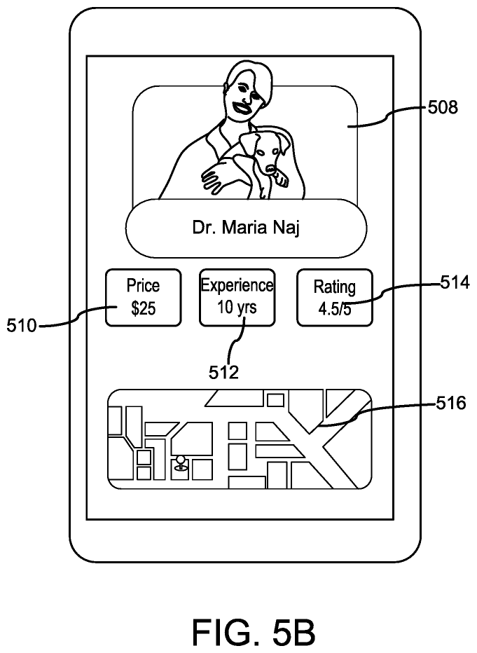

FIG. 5A illustrates another user interface allowing a user to choose one or more services for displaying veterinary professionals using the mobile application and/or website in accordance with the disclosed architecture. The user interface 500 displays the detected location 502 of the user using the device in which the application 102 is installed. Popular veterinary services 504 are displayed on the interface 500 enabling a user to choose one or more services as per their preferences. Further, based on the selected service, one or more nearby professionals 506 and/or hospitals, clinics and more are displayed for selection. In some embodiments of the present invention, the system 100 enables users to make online appointments and do online meetings with the veterinary professionals. Alternatively, in some embodiments, the system 100 provides only the detailed information and may not allow online meetings and consultations.

FIG. 5B illustrates a perspective view displaying detailed information about a selected veterinary professional in accordance with the disclosed architecture. As illustrated, the selected professional 508 is displayed along with cost 510, experience 512, and overall rating 514 of the professional. Furthermore, a digital map 516 displaying distance of the professional 508 from current location 502 of the user is also displayed for the user to make an informed decision to visit to the veterinary practitioner.

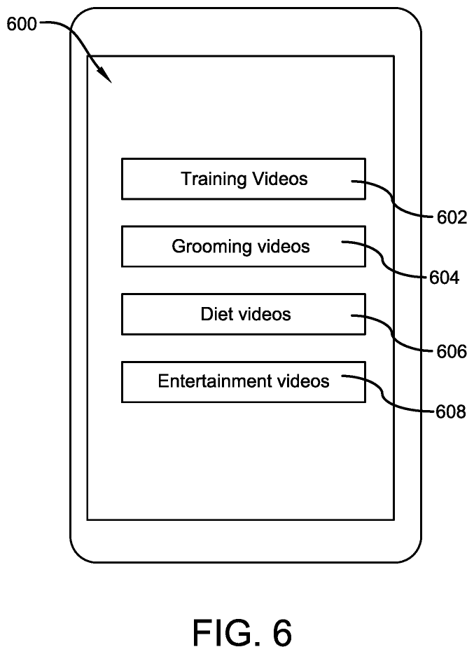

FIG. 6 illustrates another user interface designed to provide different types of audio-video content to users such as owners of pets in accordance with the disclosed architecture. The user interface 600 offers a training video icon 602 which can be selected to display a plurality of training videos for different types of pets. The training videos can be uploaded by veterinary professionals and can also be uploaded by other users. A plurality of grooming or care instruction videos can be accessed using the grooming videos icon 604. The grooming and care instruction videos can include live sessions which can be subscribed to by users based on a subscription model.

For providing information on food and diet, diet videos icon 606 on the selection thereof provides a plurality of diet videos for all types of pets and wild animals. Similarly, a plurality of entertainment videos is also stored in the server system 108 and can be accessed using the entertainment videos icon 608.

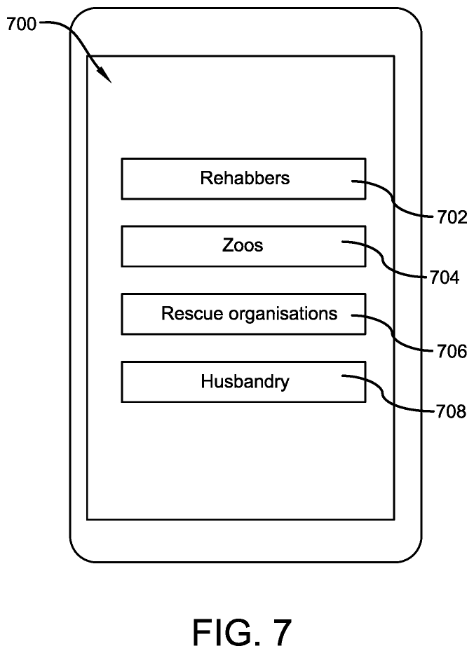

FIG. 7 illustrates another user interface configured for displaying other types of services offered by the veterinary information system of the present invention in accordance with the disclosed architecture. The user interface 700 displays a rehabbers icon 702 configured to display a list of nearby rehabbers on the selection thereof. The rehabbers can be used by people for rehabbing wild animals and more. The rehabbers in the nearby area of zip code or live location of a user are displayed. The zoo icon 704 displays a list of zoos in the nearby areas. The nearby location can be within a predefined radius of the location of the device 104 in which the application 102 or the browser 106 is accessed. Similarly, rescue organizations can be displayed upon selection of the rescue organizations icon 706 and husbandry icon 708 enables to display nearby husbandry areas for the users.

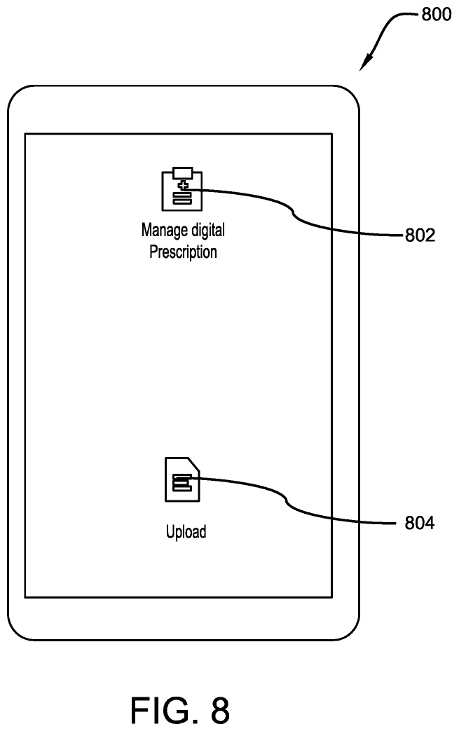

FIG. 8 illustrates a user interface offered by the animal hospitals and veterinary clinics information system allowing users to access and share digital documents of their pets with veterinary practitioners for consultation in accordance with the disclosed architecture. The user interface 800 provides a “Manage digital prescription” icon 802 for receiving and storing prescriptions from a veterinary practitioner. The prescriptions can be downloaded and can be printed as per preferences of users.

An “Upload” icon 804 enables users to upload documents, reports, videos and more in the system wherein the uploaded content is securely stored and is provided access to based on need and role basis. A veterinary practitioner consulting a pet can see the uploaded documents for an effective and accurate diagnosis and treatment, including preparation for a telehealth consult.

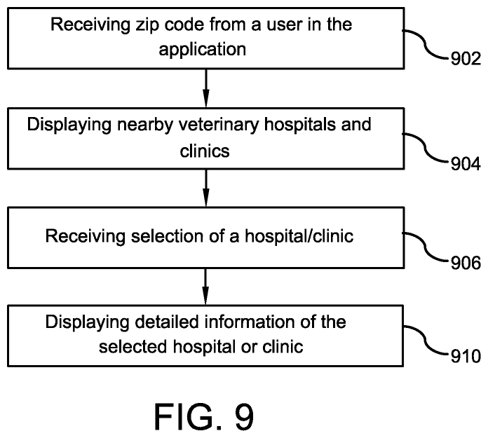

FIG. 9 illustrates a flow diagram depicting a process of displaying detailed information of relevant veterinary hospitals and clinics to a user in accordance with one embodiment of the present invention. Initially, a user enters their zip code in the application 102. The zip code is received and validated and location information such as latitude and longitude is retrieved (Step 902). Then, nearby veterinary hospitals and clinics within a certain radius utilizing the obtained coordinates are searched and displayed (Step 904). A mapping service or API, such as Google Maps API can be used to perform a location-based search for veterinary hospitals and clinics near the user's zip code. It should be noted that user profiles or preferences are considered and applied to narrow down the search results.

Then, selection of one or more hospitals, clinics, trainers, or groomers from the displayed search results is received from a user (Step 906). Finally, more detailed information as illustrated in FIGS. 4, 5A and 5B about the selected hospital, clinic, trainer, or groomer is retrieved (Step 908) and can include contact details, working hours, services offered, doctor profiles, reviews, and any other relevant data. The method can also include the additional step of displaying a notification on the mobile application indicating a higher ranked and available veterinary hospital in the geographic range of a user on selection of another veterinary hospital, clinic, trainer, or groomer by the user, wherein the notification can be based on rankings/ratings stored in the system or on a recommendation of one or more customers of the another veterinary hospital, clinic, trainer, or groomer.

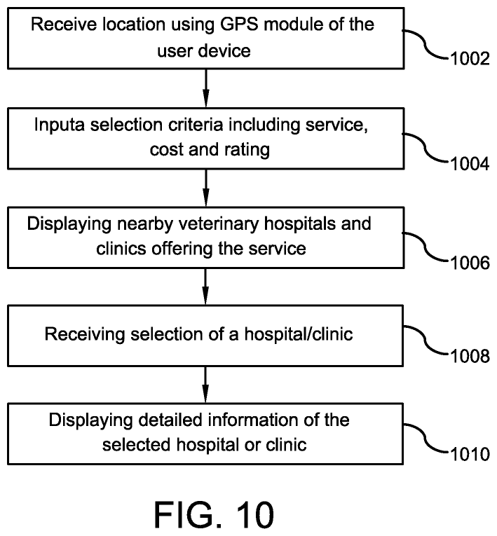

FIG. 10 illustrates another flow diagram depicting a process of displaying detailed information of relevant veterinary hospitals and clinics to a user in accordance with one embodiment of the present invention. In the present embodiment, location information is received using the GPS module of the user device in which the application 102 or the browser 106 is accessed (Step 1002). Then, a selection criteria including but not limited to service type, cost and ratings is used for specifying a search for the veterinary hospitals and clinics (Step 1004). Thereafter, the server system 108 performs a search and provides a list of nearby and relevant veterinary hospitals and clinics within a certain radius of location of the user (Step 1006). Then, selection of one or more hospitals or clinics from the displayed search results is received from a user (Step 1008). Finally, more detailed information as illustrated in FIGS. 4, 5A and 5B about the selected hospital or clinic is retrieved and displayed (Step 1010) and can include contact details, working hours, services offered, doctor profiles, reviews, and any other relevant data.

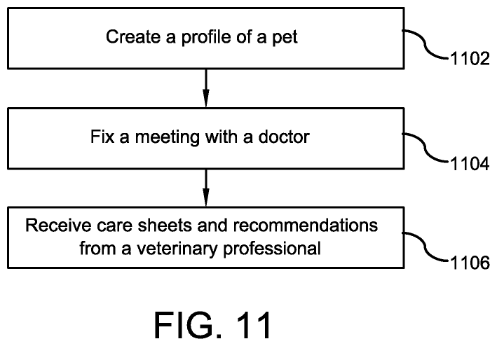

FIG. 11 illustrates a flow diagram depicting a process of receiving care sheets and recommendations from a veterinary practitioner in accordance with one embodiment of the present invention. Initially, a profile of a pet is created and is stored in the pet database 118. The profile can include past medical records, breed, height, weight and more (Step 1102). Then, an online or offline meeting (i.e., telehealth consult) is fixed with a veterinary doctor for consultation (Step 1104). The system 100 enables a user to receive care sheets and other recommendations from the consulting veterinary doctor and can also be accessed using “Manage digital prescription” icon 802 (Step 1106).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “veterinary hospitals and clinics information system”, “veterinary hospitals and clinics detailed information providing system”, “information providing system”, and “system” are interchangeable and refer to the veterinary hospitals and clinics detailed information providing system 100 of the present invention.

Notwithstanding the forgoing, the veterinary hospitals and clinics detailed information providing system 100 of the present invention can be of any suitable size and configuration as is known in the art without affecting the overall concept of the invention, provided that it accomplishes the above stated objectives. One of ordinary skill in the art will appreciate that the veterinary hospitals and clinics detailed information providing system 100 as shown in the FIGS. are for illustrative purposes only, and that many other configurations of the items organizing and identification system 100 are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An animal treatment clinic information system comprising:
a mobile computer application, a user device, a web server, an animal treatment centers database, and a server system connected through a network for information transfer between the user device and said server system;
wherein said mobile application installed on said user device having a recommendation module for providing users with detailed information on animal treatment centers;
wherein said web server receives and processes user requests;
wherein said recommendation module recommends said animal treatment centers to the user based upon received information selected from the group consisting of a user preference, a location, and a past interaction; and
further wherein said animal treatment centers database storing information on said animal treatment centers including a clinic name, an address, a contact information, a staff size, working hours, services offered, veterinarian profiles, customer reviews, and customer ratings.

2. The animal treatment clinic information system of claim 1 further comprising a pets database for storing pet information selected from the group consisting of profiles of a pet and historical data.

3. The animal treatment clinic information system of claim 2, wherein said user device selected from the group consisting of a smartphone, a tablet, and internet-enabled electronic device.

4. The animal treatment clinic information system of claim 3, wherein said network is selected from the group consisting of a public network, an internet, a private network, and a cellular communications network.

5. The animal treatment clinic information system of claim 4, wherein said server system having a network of computers and a software framework.

6. The animal treatment clinic information system of claim 5, wherein said mobile application having a user interface for displaying nearby said animal treatment centers matching a searched said clinic name.

7. The animal treatment clinic information system of claim 5, wherein said mobile application having a user interface for displaying nearby said animal treatment centers matching a searched said services offered.

8. The animal treatment clinic information system of claim 7, wherein said user interface having a digital map for displaying one or more said animal treatment centers.

9. The animal treatment clinic information system of claim 8, wherein each one of said one or more said animal treatment centers having an average of said customer ratings.

10. The animal treatment clinic information system of claim 9, wherein said animal treatment centers database storing information on said animal treatment centers further including an age of said animal treatment center and an estimate cost per visit.

11. The animal treatment clinic information system of claim 10, wherein said user interface further displaying said animal treatment center, said staff size, said age of said animal treatment center, said services offered, said estimate cost per visit, said contact information, and said rating.

12. An animal treatment clinic information system comprising:
a mobile computer application, a user device, a web server, an animal treatment centers database, and a server system connected through a network for information transfer between the user device and said server system;
wherein said mobile application installed on said user device having a recommendation module for providing users with detailed information on animal treatment centers;
wherein said web server receives and processes user requests;
wherein said recommendation module recommends said animal treatment centers to the user based upon received information selected from the group consisting of a user preference, a location, and a past interaction;
wherein said animal treatment centers database storing information on said animal treatment centers including a clinic name, an address, a contact information, a staff size, working hours, services offered, veterinarian profiles, telehealth details, customer reviews, and customer ratings; and
further wherein said veterinarian profiles include a cost, an experience, and an overall rating of a veterinarian.

13. The animal treatment clinic information system of claim 12, wherein said animal treatment centers database further storing a training video, a grooming video, a diet video, a care instruction video, and an entertainment video.

14. The animal treatment clinic information system of claim 13 wherein said mobile application having a user interface for displaying nearby said animal treatment centers matching a searched said services offered.

15. The animal treatment clinic information system of claim 14, wherein said user interface having a digital map for displaying one or more said animal treatment centers.

16. The animal treatment clinic information system of claim 15, wherein said animal treatment centers database storing information on said animal treatment centers further including an age of said animal treatment center and an estimate cost per visit.

17. The animal treatment clinic information system of claim 16, wherein said user interface further displaying said animal treatment center, said staff size, said age of said animal treatment center, said services offered, said estimate cost per visit, said contact information, and said rating.

18. An animal treatment clinic information system comprising:
a mobile computer application, a user device, a web server, an animal treatment centers database, and a server system connected through a network for information transfer between the user device and said server system;
wherein said mobile application installed on said user device having a recommendation module for providing users with detailed information on animal treatment centers;
wherein said web server receives and processes user requests;
wherein said recommendation module recommends said animal treatment centers to the user based upon received information selected from the group consisting of a user preference, a location, and a past interaction;
wherein said animal treatment centers database storing information on said animal treatment centers including a clinic name, an address, a contact information, a staff size, working hours, services offered, veterinarian profiles, telehealth details, customer reviews, and customer ratings; and
further wherein said animal treatment center is selected from the group consisting of an animal rehabber, a zoo, an animal groomer, an animal trainer, an animal rescue organization, and an animal husbandry.

19. The animal treatment clinic information system of claim 18 wherein said mobile application having a user interface for accessing and sharing digital documents with said animal treatment centers.

20. The animal treatment clinic information system of claim 19 wherein said user interface having a digital map for displaying one or more said animal treatment centers.
